(19) United States
(12) Patent Application Publication
(10) Pub. No.: US 2020/0375528 A1
(43) Pub. Date: Dec. 3, 2020

(54) Distributed Processing Intra-Oral Respiratory Monitor

(71) Applicant: Craig Thomas Flanagan, Wall Township, NJ (US)

(72) Inventor: Craig Thomas Flanagan, Wall Township, NJ (US)

(21) Appl. No.: 16/429,998

(22) Filed: Jun. 3, 2019

(51) Int. Cl. A61B 5/00; A61F 5/56; A61B 5/087

(52) U.S. Cl. CPC A61B 5/4818; A61F 5/566; A61B 5/01; A61B 5/087; A61B 5/0022; A61B 5/682

(57) ABSTRACT

Systems, methods, devices, and apparatus for monitoring intra-oral physiologic and biologic data related to sleep performance for patients with obstructive sleep apnea (OSA). In embodiments, the monitor collects data within the mouth of the patient and sends this data to a smart phone running a dedicated APP which in turn sends this data to the computer of the health care worker for evaluation. In embodiments, data collected is processed using a distributed processing arrangement and an integral scoring algorithm to produce parameters key to the diagnosis and/or monitoring of patients with OSA.

### PRIORITY CLAIM

This application claims priority to U.S. Patent Application No. 62/763,086, filed Jun. 4, 2018, entitled COMPILATION OF MANDIBULAR ADVANCEMENT TECHNOLOGIES, this application further claims priority to U.S. Patent Application No. 62/764,720, filed Aug. 16, 2018, entitled ELECTRONIC ELEMENTS OF MANDIBULAR ADVANCEMENT DEVICES, this application further claims priority to U.S. Patent Application No. 62/765,372, filed Aug. 23, 2018, entitled PATIENT COMPLIANCE/MEASUREMENT TECHNIQUES FOR MANDIBULAR ADVANCEMENT DEVICES the contents of which are incorporated by reference in its entirety.

### FIELD

The present invention relates to dental appliances and devices for the treatment and management of obstructive sleep apnea. More specifically, the present invention relates to a method of recording the sleep performance data of patients with obstructive sleep apnea and reporting this sleep performance data to health care workers.

### BACKGROUND

Obstructive sleep apnea (OSA) is a common disease affecting as much as 4% of the North American population. The cause of OSA is a partial or full collapse of the upper airway during sleep causing a reduction or cessation of breathing often accompanied by sleep arousals. Symptoms of OSA include, but are not limited to, snoring or gasping during sleep, excessive daytime sleepiness, insomnia and poor sleep quality. Untreated OSA has been associated with progression of heart disease and hypertension and other cardiovascular and neurologic sequelae.

One available treatment option for OSA is the use of so-called mandibular advancement devices (a.k.a. mandibular advancement splints, mandibular repositioning devices). Mandibular advancement devices (MADs) consist of an upper and lower jaw tray which fit over the teeth, which are operably affixed to one another, and which function to extend the lower jaw (mandible) forward a few millimeters. This forward lower jaw extension (outward extension) tends to open the airway enough that improvements are seen in occlusive breathing event frequency and severity during sleep.

A major problem with MADs is the lack of actionable clinical feedback on the appropriate amount or distance to move (titrate) the mandible during MAD fitting and therapeutic treatment. Specifically, estimates are often made by the fitting dentist or physician of a starting titration distance/setting and the MAD is fitted to the patient with this initial titration setting. Feedback on the appropriateness of the initial titration setting may consist of either subjective responses to dentist/physician questions on, for instance, sleepiness during follow-up visits or if the dentist/physician suspects that the MAD is not titrated properly, the dentist/physician may recommend a follow-up sleep study. Such sleep studies are now commonly performed at home and often consist of what is known in the art as TYPE III or IV home sleep studies (HST). These studies require that the patient wear an obtrusive instrumentation cluster during the night to collect data on sleep for further evaluation by the treating dentist/physician. TYPE III or IV home sleep studies typically provide clinical parameters useful in the diagnosis or evaluation of patient OSA symptomology such as the apnea/hypopnea index (AHI) and the respiratory disturbance index (RDI). The cost, convenience, and comfort drawbacks of these TYPE III or IV home sleep test devices are legion.

An alternate treatment strategy for OSA patients is the use of so-called CPAP devices. These devices have now evolved into several a number of technologies, but these technologies have in common that they use elevated inspired airway pressure to splint open the airway during sleep, thus preventing or reducing airway collapse. These devices typically require the use of a mask or nasal pillows as an interface to the patient to deliver the airway gas. Recent innovations in CPAP systems have included the transmission of patient sleep data back to the treating health care worker through the internet or cloud for remote patient evaluation by the treating health care worker. This has proven to be a valuable advancement.

The invention presented here extends this basic concept of remote transmission of patient data to the health care worker to the world of MADs. Specifically, sleep data during MAD nightly use is collected using a small intra-oral electronic module with integral sensors which is affixed to the MAD, and this sleep data is subsequently transmitted to the treating dentist/physician through the cloud or internet by way of a smart phone intermediary. This strategy provides a means of allowing the treating dentist/physician remote access to their patient's sleep data thereby obviating the need for the aforementioned obtrusive TYPE III or IV home sleep testing.

Methods of collecting sleep data using intra-oral electronics is described by Bradley (US2015/0112697, US2015/0169845, US2018/0261324). Bradley teaches various methods of determining if a patient is complying with a prescribed treatment regimen by verifying the patient is wearing the MAD. Bradley does not disclose methods for the monitoring and transmission of sleep metrics/parameters associated with the patient's overall sleep performance and symptomology with respect to OSA.

Kopelman teaches methods of monitoring patient sleep status using intra-oral electronics (US2016/0199215). Kopelman discloses this electronic functionality as a subsystem of a larger electro-mechanical system which advances the mandible forward automatically in response to recorded sleep data. Kopelman does not disclose methods of processing the data in order to make the data consistent with the mode of operation of TYPE III or IV home sleep test devices, and thereby Kopelman's invention is not a surrogate for TYPE III or IV home sleep test devices. For example, Kopelman does not teach the refinement of respiratory data into the apnea/hypopnea index for assessment of patient status as is common in TYPE III OR IV home sleep test devices and as described in this invention. Further, Kopelman does not disclose methods of ascertaining patient compliance with the physician/dentist prescribed treatment regimen through assessment of sensor input related to device use. Kopelman does not teach breaking out the electronics elements as a standalone device temporarily affixable to a wide variety of MADs.

Flanagan teaches methods of monitoring patient sleep status using intra-oral electronics (US2016/0324681) for the purpose of automatically advancing the mandible during sleep. Flanagan does not break the electronic components and electronics system into a separate affixable system for integration with a range of MADs and Flanagan does not teach processing the output data of sensors into a format consistent with, for instance, the apnea/hypopnea index or other parameters typically associated with TYPE III or IV home steep test devices.

Yoon teaches an electronics module which is affixed to a dental appliance predominantly for bruxism therapy (US2015/0305671) and which employs a variant of intra-oral electronics.

Dau teaches an electronics module which is attached for a form-fit dental appliance with a head kinematics sensor intended for such events as head impact (US2017/0224232).

These and other examples exist in the art but do not constitute equivalent form and functionality to the invention described herein.

### SUMMARY

Systems, methods, devices, and apparatus described herein provide for improved treatment of obstructive sleep apnea (OSA). Such improvements as described herein include the intra-oral measurement and relaying of critical metrics of sleep performance, such as apnea/hypopnea index, to the treating dentist/physician/healthcare worker in order that insight into the status of the patient's sleep performance can be understood and used as evidence undergirding future adjustments (titrations) of a mandibular advancement device (MAD) and other clinical interventions into the patients treatment protocol/regimen.

In one aspect, said critical metrics of sleep performance may include but are not limited to the apnea/hypopnea index (AHI), the respiratory disturbance index (RDI), the oxygen desaturation index (ODI), predictive analytics based on collected data, a measure of nightly patient compliance with the treatment regimen, a biologic indicator of the progression of OSA, cardiovascular metrics of the patient's state, head position based indicators of the patient's sleeping position and intra-oral audio recordings and processed audio relating to snoring and apnea events.

In one aspect, a system consisting of an intra-oral electronics module, a smart phone running a dedicated APP, and an internet or cloud connected terminal computer, smart phone or tablet displaying measured and/or processed intra-oral sleep data to the treating health care worker.

In another aspect, a system, consisting of an intra-oral electronics module in communications with a smart phone running a dedicated APP in communications with an internet or cloud connected terminal computer, smart phone or tablet, all sharing and processing data in a distributed fashion. All elements configured to collect, process and display data in a distributed fashion relating to the determination of patient compliance with the treatment regimen, and/or to the determination of patient status with respect to cardiovascular, physiologic, biologic, and/or predictive indicators of overall sleep performance.

In another aspect, an intra-oral electronics module and associated smart running a dedicated APP and an internet or cloud connected terminal computer, smart phone or tablet, all sharing and processing collected intra-oral data in a distributed fashion, consisting with respect to the intra-oral electronics, of one or more processors and memory in communications with one or more sensors. Said processor configured to run instructions allowing the collection of data, for the determination of patient compliance with the treatment regimen, and/or for the determination of patient status with respect to cardiovascular, physiologic, biologic, and/or predictive indicators of overall sleep performance.

In another aspect, a system consisting of an intra-oral electronics module in communications with a smart phone running a dedicated APP in communications with an internet or cloud connected terminal computer, smart phone or tablet. All elements configured to share and process data in a distributed fashion to produce output data for display on the internet or cloud connected terminal computer, smart phone or tablet which is substantially equivalent to the type of data produced and displayed by commercially available TYPE III and/or TYPE IV home sleep test devices (in combination with adjunct computer based sleep scoring systems).

In another aspect, a system consisting of an intra-oral electronics module in communications with a smart phone running a dedicated APP in communications with an internet or cloud connected terminal computer, smart phone or tablet. All elements configured to produce distributed processing of the data collected by the intra-oral appliance in which the bulk of data and signal processing occurs on either the smart phone running a dedicated APP or on the internet or cloud connected terminal computer or a combination thereof.

In yet another aspect, a system consisting of an intra-oral electronics module in communications with a smart phone running a dedicated APP in communications with an internet or cloud connected terminal computer, smart phone or tablet. All elements configured to produce distributed processing of the data collected by the intra-oral electronics and further configured to produce output data for display to the health care worker which includes predictive algorithms which anticipate future interventions, future patient status and future MAD performance by the analysis of past collected data.

In yet another aspect, a system consisting of an intra-oral electronics module in communications with a smart phone running a dedicated APP in communications with an internet or cloud connected terminal computer, smart phone or tablet. All elements configured to produce distributed processing of the data collected by the intra-oral electronics and further configured to produce output data for display to the health care worker which includes neural network based, or deep learning based or parametric based algorithms which obtain data similar in nature and type to the data produced by TYPE III and IV home sleep studies (and accompanying adjunct computer-based scoring systems).

In yet another aspect, a system consisting of an intra-oral electronics module in communications with a smart phone running a dedicated APP in communications with an internet or cloud connected terminal computer, smart phone or tablet. All elements configured to produce distributed processing of the data collected by the intra-oral electronics and further configured to produce output data which includes data on patient airway flow which is substantially equivalent to the flow data obtained by TYPE III and IV home sleep studies devices.

Other aspects and features of the present invention will become apparent by a review of the attached specification, figures and claims.

### BRIEF DESCRIPTION OF THE DRAWINGS

The features and advantages of the present disclosure will be more fully understood with reference to the following detailed description when taken in conjunction with the accompanying figures, wherein:

FIG. 1 depicts an embodiment of the invention which includes an electronics module and housing affixed to an oral appliance which transmits collected intra-oral physiologic or biologic data to a smart phone and which in turn transmits data to a remote program or APP through the cloud or internet for viewing by a healthcare worker.

FIG. 2 depicts an embodiment of the hardware elements only of the invention consisting of an electronics module and housing attached to the lateral aspect of the upper or maxillary tray of a mandibular advancement device.

FIG. 3 depicts an embodiment of the housing of the invention's electronic elements including posteriorly attached clips which allow the electronics module to removably attach to a mandibular advancement device.

FIG. 4 depicts the electronic circuit board of the invention which is housed in the said housing and which includes a microcontroller and ancillary integrated circuit chips and sensors.

FIG. 5 depicts the constituent elements of the said electronic circuit board housed within the said housing of the invention. Said constituent elements including sensor technologies.

FIG. 6 depicts a functional flow chart of the operation of the invention which includes, in the leftmost pane, collection of intra-oral data by the said electronics module, transfer of this data to an external smart phone APP, in the center pane, and subsequent signal processing of the data on the said external smart phone APP by a scoring algorithm, and final transfer of the processed data to a remote computer or smart phone or tablet, in the right-most pane, through the internet or the cloud. The remote computer, or optionally the smart phone APP, then runs an optional predictive analytics program on the data before displaying the data for health care workers to view.

FIG. 7 depicts the electronics module of the invention split into two separate electronics modules and attached to both lateral aspects of the mandibular advancement device to ensure better fit of the modules into the intraoral space.

FIG. 8 depicts the split modules of FIG. 7 at a functional level. The module is split into two modules with the sum total of components of the split modules being at least the equivalent of the non-split module. Two-way Bluetooth communications exists between each of the two split modules and also between each of the split modules and the external smart phone. Further, data collected and processed by each element of the split module can be shared by two-way Bluetooth communications between each of the two split modules.

FIG. 9 depicts the placement of an integral flow sensor behind the inset screen on the front of electronics module housing. Specifically, the drawing shows a slightly enlarged flow sensor and indicates that its mounting location lies behind the inset screen on the font of the electronics housing.

FIG. 10 depicts the integral flow sensor which provides a flow signal which can roughly approximate inhaled flow. Three figures indicate various levels of deconstruction of the flow sensor with the top figure showing a representation of an intact flow sensor with flow directing baffles on the left and right lateral aspect of the sensor. The figure to the lower left indicates the appearance of the flow sensor with the baffles removed. The figure to the lower right indicates the appearance of the flow sensor with the front cover removed and the integral flow chamber and flow transducer (pressure, gas flow or gas humidity) exposed on the rear aspect of the sensor.

FIG. 11 depicts gas temperature change as measured by a temperature sensor within the housing of the flow sensor as a function of increasing (constant) gas flow.

FIG. 12 depicts the supplemental use of the exposed metallic charge stubs 12 which are primarily used as a cathodic and anodic contact points for an external magnetic charger. The supplemental use includes a measurement of electrical resistance between the charge stubs as a means of determining if the patient is using the device (e.g. as a measure of patient treatment compliance).

FIG. 13 depicts the hardware and software elements of the head position sensing configuration of the invention including wireless transmission of head orientation data to an external smart phone APP.

FIG. 14 is a flow chart showing the scheme of collection and storage of three-dimensional head orientation data for the invention.

FIG. 15 is a flow chart of an algorithmic strategy for obtaining a sleep restlessness factor or measure and categorizing sleep restlessness during a period in time as being low, medium or high.

FIG. 16 is a flow chart of an algorithmic strategy for determining the frequency of rapid head movements during a period in time as being low, medium or high.

FIG. 17 depicts two plots showing the juxtaposition of a head orientation data plot above a plot of snoring activity as a means of gaining insight into position dependent sleep apnea. Such a juxtaposition potentially leading to improved diagnosis of pathophysiologies or disease states such as supine dependent sleep apnea.

FIG. 18 depicts an artificial neural network (ANN) configuration in which input sensor activity feeds through nodes to obtain clinically valuable output parameters such as apnea/hypopnea index.

FIG. 19 depicts an alternate (to FIG. 18) means of using a parametric algorithm in combination with sensor input to obtain estimates of clinically valuable parameters such as the apnea/hypopnea index.

FIG. 20 depicts training and implementation configurations for a signal processing strategy to implement active noise cancellation (ANC) in an intra-oral device to reduce or remove sounds such as snoring.

FIG. 21 depicts screen display elements of the dedicated smart phone APP which the electronics module of the invention sends data to and which, itself, transfers information to the terminal computer, smart phone or tablet of the health care workers computer.

FIG. 22 depicts one embodiment of a predictive analytics algorithm integrated into the software on the health care workers terminal computer, smart phone or tablet.

### DETAILED DESCRIPTION

An improved understanding of the advantages, features, and elements of the present disclosure will be obtained by reference to the following detailed description. This description sets forth descriptive and illustrative embodiments, for which the principles of the present disclosure will become clear.

Current methods of determining the efficacy of patient treatment with respect to mandibular advancement therapy for OSA patients include the use of home sleep monitors such as TYPE III and IV home sleep devices. These devices take disparate data from a variety of sensor sources and integrate this raw data into clinically meaningful data for evaluation of patient sleep performance. Such devices are obtrusive and worn at night upon physician recommendation to obtain insight into sleep performance, ostensibly for improved titration of the mandibular advancement devices. The invention described herein produces measures of clinical importance similar to TYPE III and IV sleep monitors but in a significantly less obtrusive way.

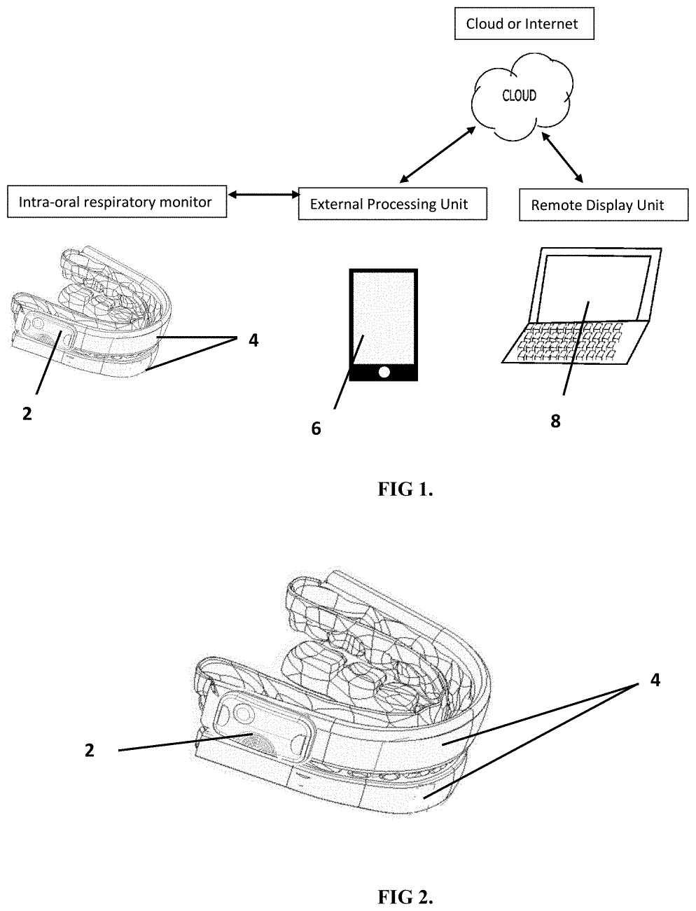

Specifically, the invention herein uses three analytically integrated components, an intra-oral electronics module, a smart phone running a dedicated APP and a terminal device such as a computer, smart phone or table which displays calculated sleep metrics for health care workers to see. FIG. 1 shows the intra-oral electronics module 2 which is removably or permanently attached to a mandibular advancement device 4. The electronics module sends data which it records with its on-board sensors systems to an external smart phone 6 APP by wired or wireless means such as Bluetooth and ultimately to a remote computer, smart phone or tablet 8 via the internet or cloud.

The invention presented here uses intra-oral sensing as a means of determining clinical parameters such as the Apnea/Hypopnea Index which is a function performed by current devices known in the art as home sleep test (HST) devices which are available commercially and are divided by capability or sensing means as TYPE I, II, III, and IV devices. This invention provides functionality roughly equivalent to TYPE III or IV HST devices in that, as with these monitors, it uses a small number of sensors to obtain clinically meaningful information on the sleep status of patients throughout the night including sleep apnea frequency/severity.

In embodiments, display of the nightly sleep data collected by the intra-oral electronics module can occur (for the patient to view) on the smart phone APP 6, or (for the health care worker to view) on the terminal device 8. Either way, the data is transmitted in an encrypted fashion to prevent potential loss of private data.

In some embodiments, the bulk or majority of data or signal processing is performed by either the smart phone 6 APP or by the terminal program 8 or by a combination thereof. It is understood that the electronics module is only responsible for collection of sensor data, mild processing if any, and relay of that data (in essentially raw form) to the smart phone APP or terminal program where it is processed and this processing comprises the bulk of data and/or signal processing performed on the invention.

In some embodiments, the electronics module may be configured with a high-power processor to itself perform the bulk of data and/or signal processing. It should be noted that the drawback of a high-power processor is heavy current consumption which requires larger batteries, and this is suboptimal in the space constrained intra-oral environment.

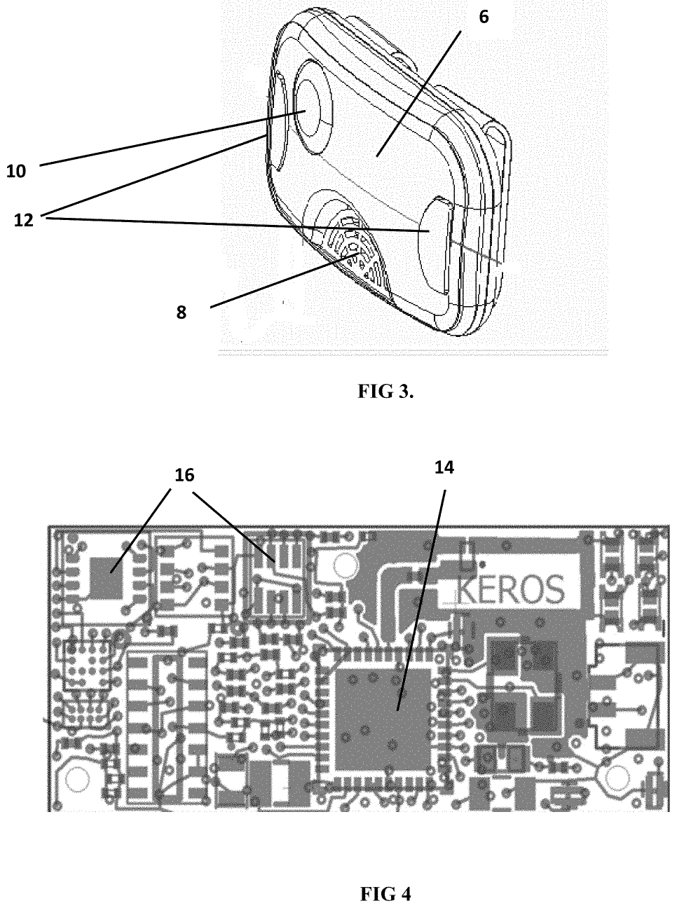

In embodiments, FIG. 2 depicts a close-up of the electronics module 2 as connected to the maxillary tray of a mandibular advancement device 4. It is understood that this mounting location is not the only potential mounting location which might be used. The mounting may be achieved by a clip, a magnet, a snap or a similar mounting mechanism which removably and temporarily affixes the invention to the oral appliance. Mounting may also be achieved by permanent mounting means such as glue or rivets or fasteners. The removable aspect of mounting is highly advantageous for multiple reasons, first, removable mounting allows for batteries and electrical components to be more easily serviced, second, removable mounting may allow for easier device (battery) charging, third, removable mounting allows the invention to be removed from the patients mouth when device use is not essential thereby improving overall comfort of the patient due to the space requirements imposed by the electronics module in a space limited or constrained (intra-oral) environment and fourth, removably mounting the invention ensures that the invention can be more easily manufactured. For instance, some prior art referenced here does not advantage itself of this removability element. By example, Dau's (US2017/0224232) electronics elements are form fit to a 3-D scan of the teeth, Dau details the extensive manufacturing requirements this integration imposes while losing the advantages just outlined of removability, serviceability and patient comfort.

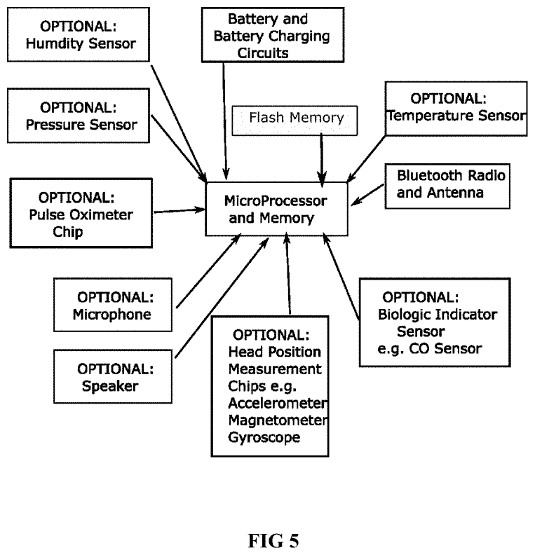

In embodiments, FIG. 3 depicts a close-up of the electronics module showing the various elements of one embodiment of the housing of this module. The module housing includes a body 6 which may be of clam shell type with integral O-rings or gaskets to prevent water ingress, or similar configuration which prevents water ingress. Integral to this housing is a vent 8 which allows for gas entry into the interior aspects of the housing while preventing bulk water ingress by means of a PTFE hydrophobic membrane media or similar media which allows for gas transit while preventing bulk water/sputum/saliva ingress. The housing includes a lens 10 which allows the (optional) pulse oximeter chip optical access to the buccal mucosa so that data on heart rate and blood oxygen saturation can be obtained. The lens is assumed clear or optionally has optical properties which filter out particular wavelengths of light. The lens is fitted by means which produces a water-tight seal so that water cannot enter the housing. This may be achieved by means of O-rings, glue, interference fit, or similar technique. Two metallic stubs 12 penetrate the skin of the housing and these too are fitted so as to prevent water ingress. The stubs are connected to the internal electronics circuit boards and are used as cathodic and anodic contact points for an external magnetic or contact-based DC charger. It will be seen that these stubs may also be used in some embodiments as a contact point on the patient for electrical resistance-based determination of patient use of the device (a.k.a. compliance). FIG. 3 should be considered an exemplary embodiment of the housing of the electronics module and other similar housing configurations are possible and should be considered within the scope of this invention.

In some embodiments, the electronics module may be protected from liquid ingress using a biocompatible polymer or elastomer.

In embodiments, FIG. 4 depicts the internal electronic circuit board housed within the electronics module housing. The circuit board having at least one integral microprocessor 14 and one or more integral sensors 16.

In some embodiments, the microprocessor may be substituted with a microcontroller, a field programmable gate array (FPGA), an application specific integrated chip (ASIC) or a similar programmable digital logic processing device.

One embodiment of the electronics elements of the invention is shown in FIG. 5. These electronics elements being understood to include the circuit board, integrated circuits and sensors which are housed in the housing of the intra-oral electronics module. The electronics elements include a microprocessor and memory, both volatile and non-volatile (e.g. flash memory) which serves as the central data processing element of the circuit. Data is collected from the various sensor systems by the microprocessor and stored either in volatile or non-volatile memory. Data, once collected, is transferred by a wired or wireless means, such as by the Bluetooth radio and antenna to the external smart phone APP.

In embodiments, the microprocessor uses input from an external accelerometer to both put the electronics to sleep as well as to wake the electronics based on device movement. This serves two key purposes, battery preservation and ease of use. With respect to battery preservation, the device will fall asleep due to lack of movement, for instance when the electronics module is placed on a table during the day when the device is not in use. This preserves battery life. Second, the wakeup and sleep functionality provided by the accelerometer allows the electronics module to function without an external power switch. This is advantageous as it prevents accidental misuse by the patient, who might forget to turn the device on, for instance.

In embodiments, the electronics module includes a chipset which allows for the determination of patient head position/orientation FIG. 5. This may include a three-dimensional accelerometer, a three-dimensional inertial measurement unit, a three-dimensional gyroscope, a magnetometer, a tilt sensor, an altitude sensor, or similar sensors which provide data of value in calculating the orientation in space of the user or wearer of said electronics module. Said integrated circuits may, by example, include an AK09915 magnetometer (AKM Semiconductor Inc), a BMI160 (Bosche Sensortech) inertial measurement or similar commercially available integrated circuits which are sensitive to tilt or rotation, or inertial characteristics, or magnetic orientation (compass) or acceleration.

In embodiments, a sensor element which is sensitive to a biologic indicator of patient status is present in the electronics module, FIG. 5. It is known in the art that certain gaseous molecules present in the breath are potential surrogates for the severity of sleep apnea. Such molecules include nitric oxide, carbon monoxide, ethylbenzene, p-xylene, phenylacetic acid, and nonane. Specifically, these molecules have been seen to vary in concentration with sleep apnea severity. These molecules will be referred to here as biologic indicators of disease severity.

In some embodiments, a MEMS or amperometric or miniature fuel cell or other type of small sensor is used to obtain discrete values of the biologic indicator concentration in the breath. For example, clinically significant levels of carbon monoxide in the exhaled breath of approximately 2 to 5 PPM can provide clinical insight into obstructive sleep apnea disease state severity and such ranges are within the measurable ranges of off-the-shelf carbon monoxide sensors.

In some embodiments, the invention includes the use of a small gas sensor, FIG. 5, to measure the concentration of nitric oxide, carbon monoxide, ethylbenzene, p-xylene, phenylacetic acid, or nonane and other biologic indicators of disease severity. Such gas sensor monitoring being of value for insight into obstructive sleep apnea disease progression, severity or amelioration.

In some embodiments, FIG. 5, a humidity sensor, or a temperature sensor, or a pressure sensor may be used as the basis for a flow sensor to determine the breathing gas flow of the patient's breathing. Such a sensor configuration will be described elsewhere in this document.

In some embodiments a pulse oximeter chip may be integrated into the electronics module FIG. 5. The pulse oximeter being used to obtain discrete readings of heart rate and blood oxygen saturation.

In some embodiments, FIG. 5, a microphone may be integrated into the electronics module. The microphone providing insight into patient condition by tracking such sounds as snoring, coughing, occlusive airway sounds and breathing. The microphone signal may be processed using spectrally discriminatory filtering techniques such as low pass, high pass and band pass filters to isolate important spectral constituents helpful in extracting or identifying particular sounds.

In some embodiments, FIG. 5, a speaker may be integrated into the electronics module. The speaker may be employed to wake the patient due to, for instance, a sensor reading which would indicate problematic and/or dangerous sleep state or performance, such as a low SpO2 (blood oxygen saturation) reading. In some embodiments, the speaker may be combined functionally with the microphone and the microprocessor to produce an active noise cancellation (ANC) system to cancel out or significantly reduce the volume of snoring and other sounds during sleep.

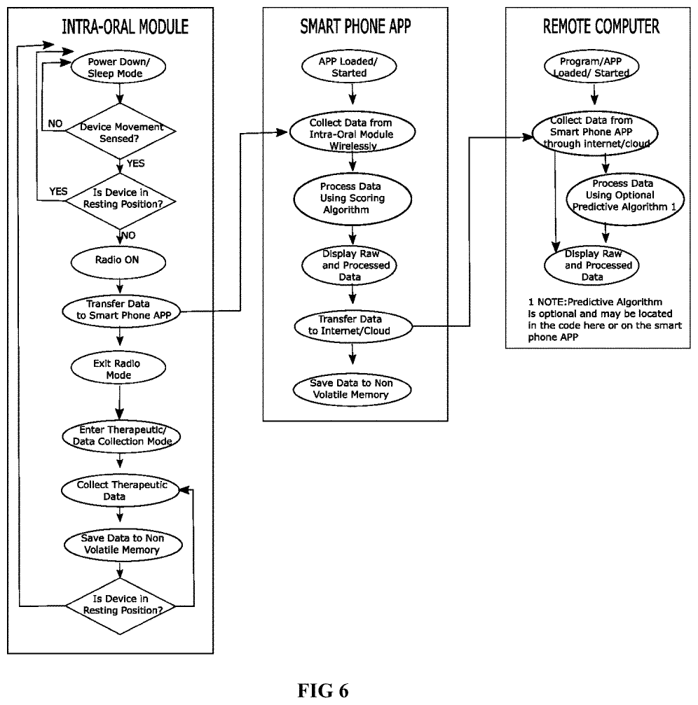

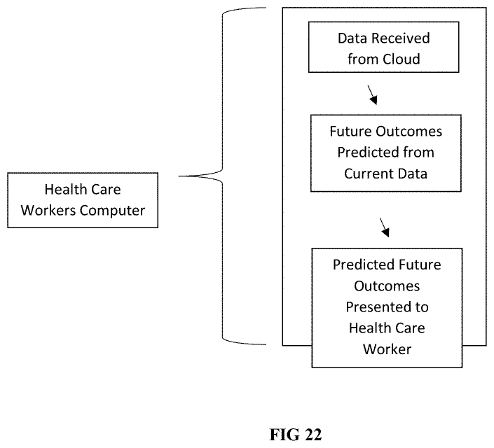

In embodiments, the processing of data by the system, may include program flow as depicted in FIG. 6. Such exemplary program flow includes maintenance of proper on/off functionality of the electronics module, proper and timely control of the Bluetooth radio by the electronics module, and proper collection and transference of data by the electronics module to the smart phone running a dedicated APP. The smart phone APP then, in the exemplary embodiment shown, processes the data using a “scoring algorithm” which functions to process raw data collected from the electronics module and turn it into clinically meaningful data such as the apnea/hypopnea index before transferring this processed data to the remote computer (or terminal computer) where it may be further processed using a predictive analytics algorithm FIG. 22. It is understood that the scoring algorithm and the predictive analytics algorithm may be co-located on the smart phone APP, co-located on the remote computer (terminal computer) or the predictive analytics algorithm may be eliminated altogether.

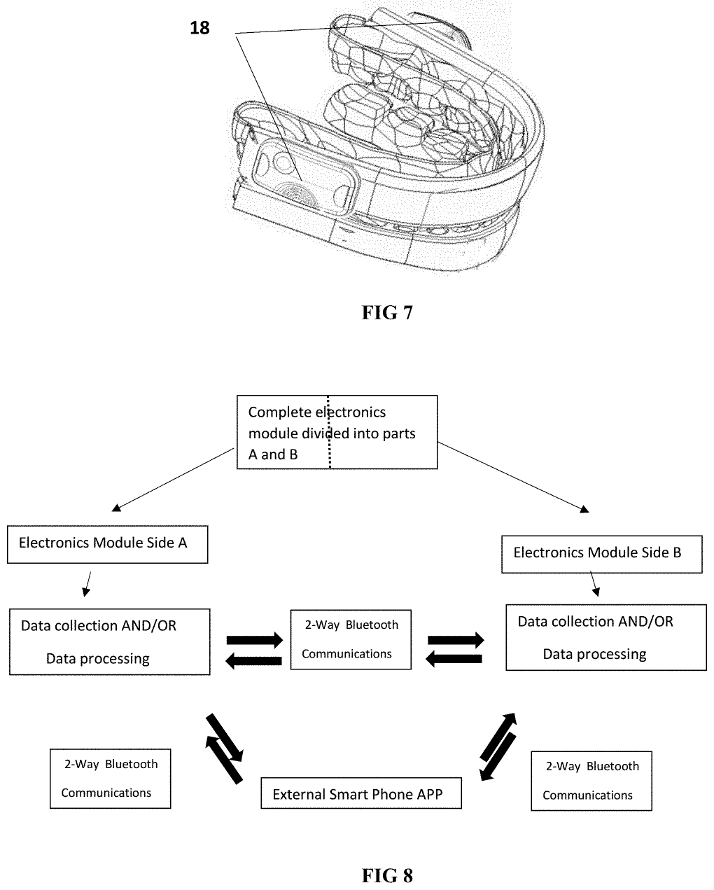

In some embodiments, the electronics module of the invention may be split into two or more elements 18 as depicted in FIG. 7. The sum total of the electronic components on these two or more split electronics modules is understood to be the equivalent of the individual module with respect to sensor technologies and as described in FIG. 5. However, each split module would be understood to have its own dedicated processor and processor support electronics (e.g. crystals and memory). The splitting of the electronics module being advantageous to ensure that the electronics modules fits more easily or comfortably into the highly limited intraoral space.

In some embodiments, FIG. 8, the splitting of the electronics module is implemented such that each split electronics module can interact via Bluetooth (or other wired or wireless means) with other split elements of the electronics module and each split element of the electronics module can interact via Bluetooth (or other wired or wireless means) with the external smart phone.

In some embodiments, the split elements would be treated as nodes of a mesh network. In such a configuration, the nodes would connect non-hierarchically and/or directly and/or dynamically to each other. The nodes are understood to be capable of self-configuring and/or self-organizing (self-forming) and/or self-healing and/or self-optimizing. The nodes may be configured as well with multi-hop connectivity.

Commercially available TYPE III and IV HST devices for diagnosing and/or determining sleep apnea severity often rely on one of two types of sensors to determine breathing air flow, an oro-nasal thermal sensor or nasal pressure sensor. It should be noted that sleep belts (plethysmography) for thoracoabdominal movement are also often used as well. These sensors are used to obtain an indication of gas flow amplitude and direction and are further used to determine if there has been a cessation or slow down of inhaled gas flow, a condition synonymous with sleep apnea. Neither of these types of sensors provide a high degree of accuracy of inhaled gas flow and thus one might consider the output signal from these sensors as being pseudo-proportionate or loosely proportionate to actual inhaled flow magnitude.

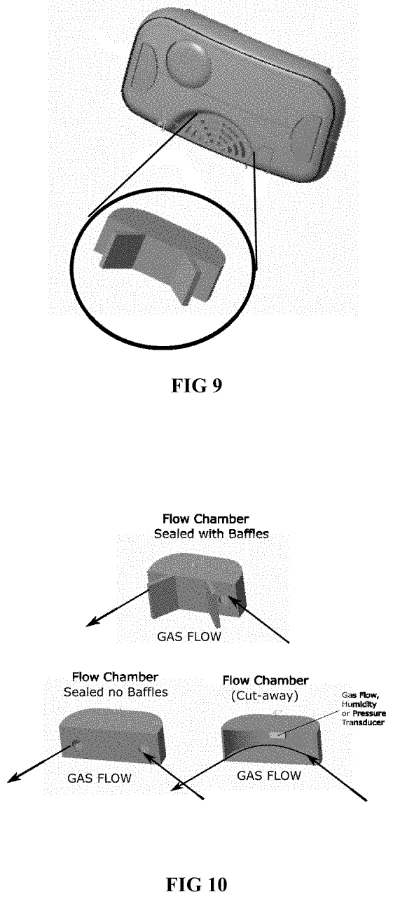

In embodiments, FIG. 9 depicts a unique sensor employed in the invention which consists of a micro-mixing chamber into which gas flow is directed using baffling. Such a sensor configuration produces a low accuracy proportionate indicator of inhaled flow. The accuracy of such a sensor being sufficient in order that characterizations of apnea events by this invention can be determined. Such a configuration rivaling the accuracy of oro-nasal thermal sensors or nasal pressure sensors and thus being adequate to the task of sensing patient airway flow.

In some embodiments the flow sensor FIG. 9 is incorporated within the electronics module housing to provide a means of sensing breathing gas flow.

In embodiments, FIG. 10 depicts the piece-wise disassembly of the flow sensor starting with the upper pane which shows the fully assembled flow sensor, the lower left pane which shows the flow sensor with the baffling removed, and the lower right pane which shows the flow sensor with the front cover removed exposing the pressure, humidity or temperature sensor housed within the sampling or mixing chamber.

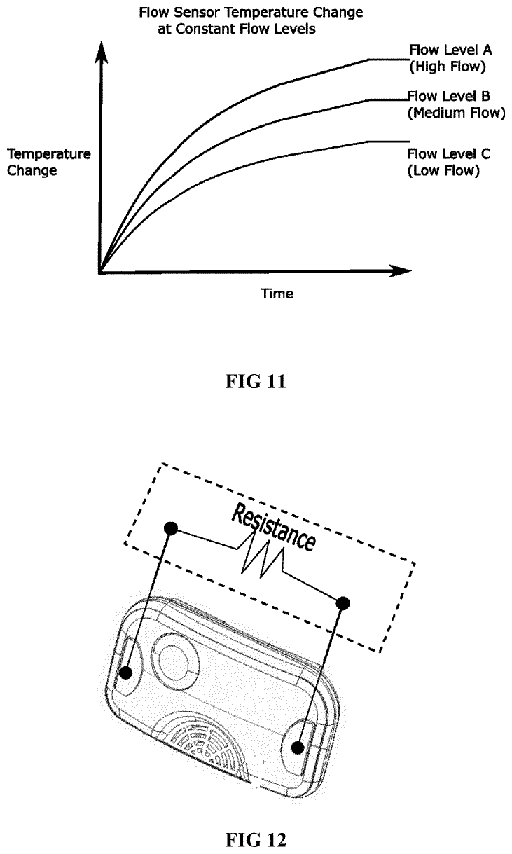

In embodiments, the gas sensor operates by using baffling to force a small stream of inhaled or exhaled gas flow through its inner chamber thereby directing the breathing gas flow into the inner chamber. Within the chamber is a temperature sensor but it is understood that the temperature sensor could be replaced with a pressure sensor or a humidity sensor. On inhalation, the gas temperature within the mouth quickly changes from the temperature of the gas at end exhalation (approximately 35-37 degrees C.) to room temperature as this end exhalation gas is quickly replaced with room temperature air within the mouth as air streams into the lungs. FIG. 11 provides a view of the (absolute value of) temperature change which occurs within the chamber as a function of constant inhaled gas flow. The derivative of this signal, or the value of the rate of change of temperature is roughly proportionate to gas flow and can be converted to gas flow by using an empirically derived lookup table in the software code which provides estimates of the conversion of rate of temperature change information to gas flow.

In some embodiments of the invention, patient compliance with the treatment regime is determined by monitoring the use of the electronics module during sleep. Such monitoring can take many forms and are discussed here. Firstly, FIG. 12 depicts a method of determining if the patient is wearing the device. FIG. 12 depicts a resistive check during operation between the charge stubs 12 by the internal electronics within the electronics module. The resistance between the terminals will change in a quantifiable way once the device is worn due to the electrical conductivity path available between the charge stubs being altered by the moisture in the mouth and by the contact of the stubs with the skin of the inner cheek or buccal mucosa. This resistance change can be monitored and used as an indicator of patient compliance or non-compliance with the treatment regime.

In some embodiments of the invention, patient compliance with the treatment regimen may be monitored by changes in signals obtained by the on-board sensor technologies in the electronics. Patient use of the device is synonymous with patient compliance given that patient use of the device meets the treating health care workers requirements for device use. Examples are now listed and it should be noted that while individual measures of patient compliance (e.g. meeting criterion “c” below should be considered sufficient for assessing patient compliance, it should also be understood that any combination of the criteria below could also be considered an indicator of patient compliance (e.g. combining criterion “d” and criterion “f”):

In embodiments, modes of determining patient compliance are now listed to include movement indicators used as a means of assessing patient compliance. This includes movement indicators as measured using integral head orientation sensing electronics (note: head movement is assumed to be the equivalent of electronics module movement when device is being worn).
a. Intermittent head rotation about the patient's vertical, long or longitudinal axis above a predefined event threshold value.
b. Movement of head above a predefined threshold value for translational or rotational movement. With rate of translational movement in the X, Y or Z axis or combination thereof and rate of rotational movement along the X, Y or Z axis or combination thereof
c. Oscillatory head movement within a temporal band defined by typical human breathing patterns (e.g. back and forth head movement in the Z axis direction at a frequency of 0.7 Hz).
d. Head movement above a power spectral density threshold within spectral bands associated with human breathing or snoring.
e. Oscillation of head movement-based power spectral densities (measured within spectral bands associated with human snoring) with oscillation occurring at frequencies associated with human breathing.
f. Movement of the head/device away (for X hours) from the resting position that the device has occupied for the previous Y hours. Note: values of X and Y are predefined.

In embodiments, modes of determining patient compliance are now listed to include gas flow indicators used as a means of assessing patient compliance. This includes gas flow indicators as measured using the integral flow sensor of the invention which is described herein as being either a pressure, humidity or temperature sensor disposed within a sampling chamber:
a. Oscillatory flow as measured by the flow sensor within a frequency band associated with human breathing over a predefined time threshold (e.g. oscillatory flow measured at 0.6 Hz for 6 minutes).
b. Inhaled or exhaled flow as measured by the flow sensor above a predefined threshold value.

In embodiments, modes of determining patient compliance are now listed to include sound indicators used as a means of assessing patient compliance. This includes sound indicators as measured using the integral microphone of the invention:
a. Oscillatory sound power spectral densities as measured at frequencies bands associated with human snoring and oscillating at frequencies associated with human breathing.
b. Power spectral densities above a threshold as measured at frequency bands associated with human snoring.
c. Oscillatory sound power spectral densities as measured at frequencies bands associated with human breathing sounds and oscillating at frequencies associated with human breathing.
d. Power spectral densities above a threshold value as measured at frequency bands associated with human coughing.

In embodiments, modes of determining patient compliance are now listed to include pulse oximetry indicators used as a means of assessing patient compliance. This includes pulse oximeter data indicators as measured using an integral pulse-oximeter chip:
a. Heart rate measurement within predefined limits associated with human rates for a predefined duration (e.g. heart rate measured at 68 BPM for 5 minutes).
b. SpO2 measurements within predefined limits associated with human blood oxygen saturation levels for a predefined duration (e.g. SpO2 measured at 92% for 7 minutes).

In embodiments, modes of determining patient compliance are now listed to include mean humidity indicators used as a means of assessing patient compliance. This includes mean humidity data indicators as measured by taking the mean value of the sensed humidity.
a. Mean humidity (absolute and relative) level changes above a threshold value for a predefined duration (e.g. mean Rh seen to change from 38% to 62% for 11 minutes).

In embodiments, modes of determining patient compliance are now listed to include mean temperature indicators used as a means of assessing patient compliance. This includes mean temperature data indicators as measured by taking the mean value of the sensed temperature.
a. Mean temperature value changes above a threshold value for a predefined duration (e.g. mean temperature seen to change from 25 degrees C. to 31 degrees C. for 4 minutes).

In embodiments, modes of determining patient compliance are now listed to include biologic indicators used as a means of assessing patient compliance. This includes biologic indicator data as measured by taking a periodic value of a biologic indicator.
a. Value of measured biologic indicator seen to change above a threshold value for a predefined duration (e.g. Carbon Monoxide level seen to increase from 0.2 PPM to 3.1 PPM for 16 minutes).

In embodiments, a depiction of the electronic circuit board of the invention is shown in FIG. 4. The electronic circuit board contains integrated circuits which make head orientation sensing possible. This includes a digital signal processing chip 14 such as a microprocessor, microcontroller, field programmable gate array (FPGA), an application specific integrated chip (ASIC) or a similar programmable digital logic processing device. Also included are ancillary integrated circuit chips 16 and as noted these integrated circuit chips may include a three-dimensional accelerometer, a three-dimensional inertial measurement unit, a three-dimensional gyroscope, a magnetometer, a tilt sensor, an altitude sensor, or similar sensors which provide data of value in calculating the orientation in space of the user or wearer of said electronics module. Said integrated circuits may, by example, include an AK09915 magnetometer (AKM Semiconductor Inc), a BMI160 (Bosche Sensortech) inertial measurement unit or similar commercially available integrated circuits which are sensitive to tilt or rotation, or inertial characteristics, or magnetic orientation (compass) or acceleration.

In embodiments, the orientation of the invention with respect to its attached oral appliance is fixed and the orientation of the oral appliance when worn by the patient is also fixed relative to overall head position. Therefore, data obtained from orientation-based sensors do not need to accommodate any variation in head orientation with respect to mandibular advancement device orientation as their respective orientations are fixed with respect to each other. Thus, there needs to be only one orientation calibration, and this will suffice.

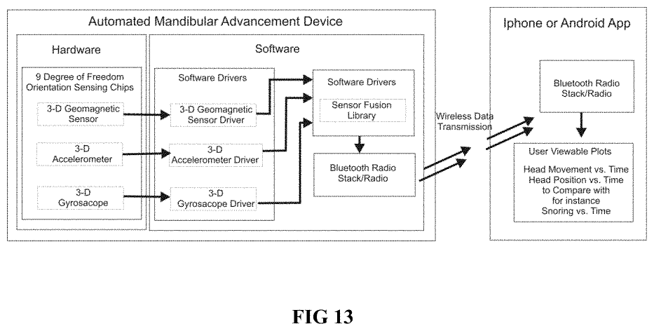

In embodiments of this invention, data is collected by the integral orientation sensing integrated circuit chips and sent to a microcontroller or similar digital processing chip as shown in FIG. 5. Each orientation sensitive integrated circuit chip FIG. 13 then has a specific software driver associated with it which processes the raw data collected by the digital processing chip and this data is further processed by a software component, often referred to in the art as a sensor fusion library, which takes the composite orientation data and converts it to Euler angles, quaternions or matrix algebra representations of orientation in 3-D space. Such a representation may be timestamped or sequentially stored in order that head position vs. time is known or may even be subjected to initial mathematical operations such as differentiation with time or integration with time to obtain insight into movement characteristics not readily available or observable in a time stamped sequence of head position data.

In embodiments, the configuration of FIGS. 4, 5 and 13 includes a 9 degree-of-freedom sensing means consisting of multiple ICs or chips (or potentially a future single chip) which reports X-Y-Z accelerometer data, reports X-Y-Z gyroscopic measurement and reports X-Y-Z geomagnetic data. Specifically, the following chip set may be used and should be considered exemplary as other chip sets which perform similar functions are now available: 1) 3-D Geomagnetic Sensor (3-D digital Compass) Bosch BMM150, 2) 3-D Gyroscope, Integral to Bosch BMI160 3) 3-D Accelerometer, Integral to Bosch BMI160. These chips, when used in combination, allow a device to assess its orientation in 3-D space. In the case of this chipset, there exists three software drivers which allow a calling program to obtain, in real-time, geomagnetic, gyroscopic and accelerometric data. These data are then fed into a “data fusion” library which fuses the data to obtain a three dimensional representation in time which can be, for instance, represented by Euler angles which represent the rotation of a body about a fixed origin, or by for instance quaternions which are an effective means of representing the 3D orientation of the object in space. Other representations of three-dimensional orientation are known in the literature and such representations are simply additional means of presenting the data discussed here and should be considered within the scope of this invention.

In embodiments, FIG. 13 also shows the wireless transmission of head orientation data from the invention to a smart phone APP which it can be plotted in a way which provides clinical or patient oriented insight into sleep patterns or sleep behavior or patient status or disease status or disease progression or disease diagnosis. Such wireless means may by example consist of a BlueTooth transceiver and a BlueTooth antenna for transmission and receipt of data to and from external devices such as smart phones and tablets.

In embodiments of this invention, digital signal processing techniques employed on the head position data are employed in one or more of three locations to process the orientation data coming from said orientation sensitive integrated circuits. These locations include digital signal processing on the electronics module, digital signal processing on an external smart phone (with dedicated APP) or external tablet or computer (with dedicated APP) which is in wireless communications with the electronics module, or finally digital signal processing may occur on a device with which the dedicated APP is in communications with such as a cloud connected program on a health care workers computer or tablet or smart phone.

In embodiments of the invention, said digital signal processing techniques may include refinement or synthesis of disparate sensor information into a 3-D representation of head position such as a Euler angles, quaternions or matrix algebra representations. Such a representation may be timestamped or sequentially stored in order that head position vs. time is known or may even be subjected to further mathematical operations such as differentiation with time or integration with time to obtain insight into movement characteristics not readily available or observable in a time stamped sequence of head position data.

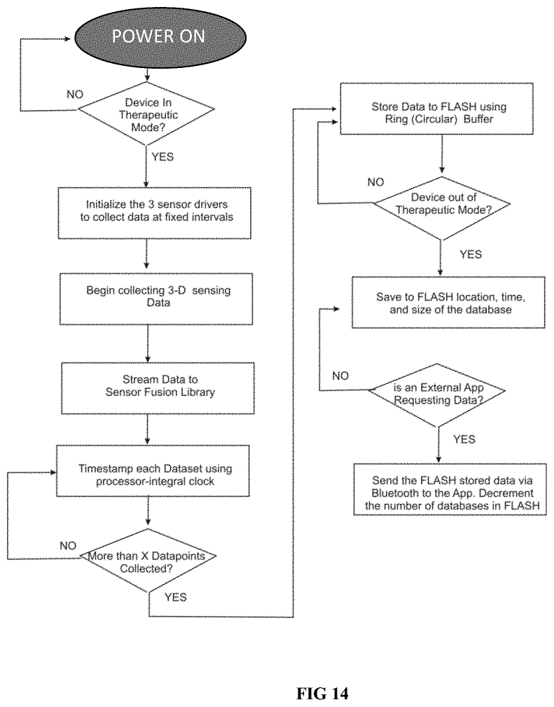

In embodiments of the invention, FIG. 14 depicts a flow chart representation of the processing of the head position data including the timestamping of the data, the saving of the data to non-volatile FLASH memory and the transmission of this data to an external smart phone APP.

In embodiments of the invention, the head position data is further processed by an algorithm which uncovers or illustrates aspects or characteristics of head position and movement which could be beneficial relative to uncovering or illustrating or identifying aspects of the medical condition of the wearer, or aspects of the behavior of the wearer or aspects of the treatment methodology of the treating health care worker, for instance, in creating an appropriate or effective treatment regimen.

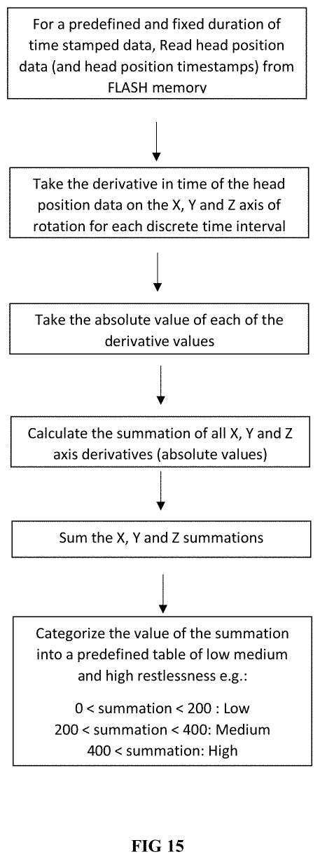

In embodiments of the invention, post processing of head position data may include measures of patient restlessness during the sleep period. Such information may be clinically valuable as a surrogate for patient awakening or other patient states. FIG. 15 shows an algorithm which uses differentiation in time to determine how much the patient is moving over a specific time period. Differentiation occurs on each new discrete value of head position obtained and each of three orientations in space (either rotational or translational) and a summation of these differentiations takes place to obtain a measure of the overall amount of movement over a time period for characterization of patient restlessness and low, medium and high.

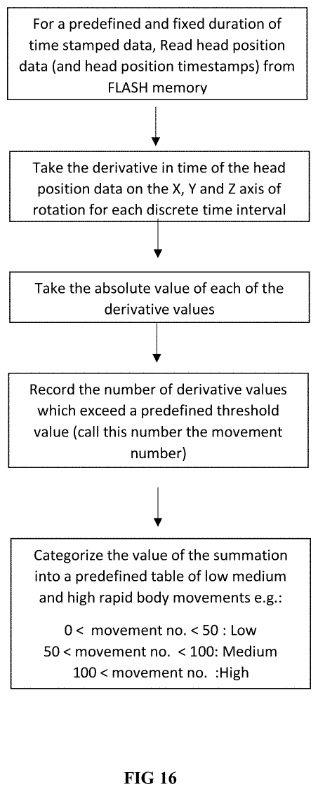

In embodiments of the invention, the relative degree of rapid patient head movements may also be of value clinically and as such the invention includes an algorithm which measures of the degree of rapid head movement over a period of time as shown in FIG. 16. The calculations made include a determination of the number of events which exceed a rapid movement threshold. Results are categorized into low, medium and high.

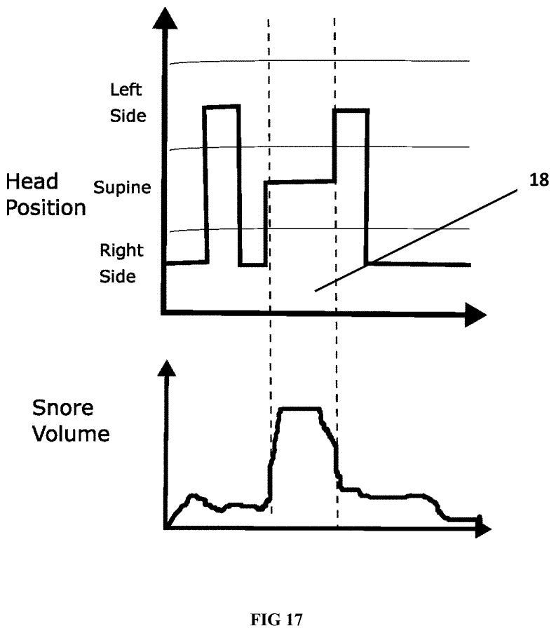

In embodiments of the invention and as another example, it is known in the art that obstructive sleep apnea often includes a positional component in which, for instance, the sleep apnea patient suffers increased sleep occlusive breathing while in a particular position such as sleeping on the back. This condition is often referred to as supine-dependent sleep apnea. The invention described here, when combined with additional sensing technology which can determine breathing characteristics or snoring sounds for instance, can be combined with the head position information to obtain clinical insight into possible position dependencies in the severity of the sleep apnea. Such a juxtaposition of data as displayed on a smart phone APP is shown in FIG. 17 wherein it is shown in 18 that particular periods in time may correlate head position with increased snoring, for instance, allowing clinical insight into head position on disease processes or expressions thereof. Other juxtapositions of data and data plots may be envisioned and should be considered within the scope of this invention.

In embodiments, the calculation of clinically valuable parameters with respect to identification and quantification of OSA such as the apnea/hypopnea index (AHI), or the respiratory disturbance index (RDI) are calculated either on the smart phone APP or on the terminal computer, smart phone or table used by the health care worker.

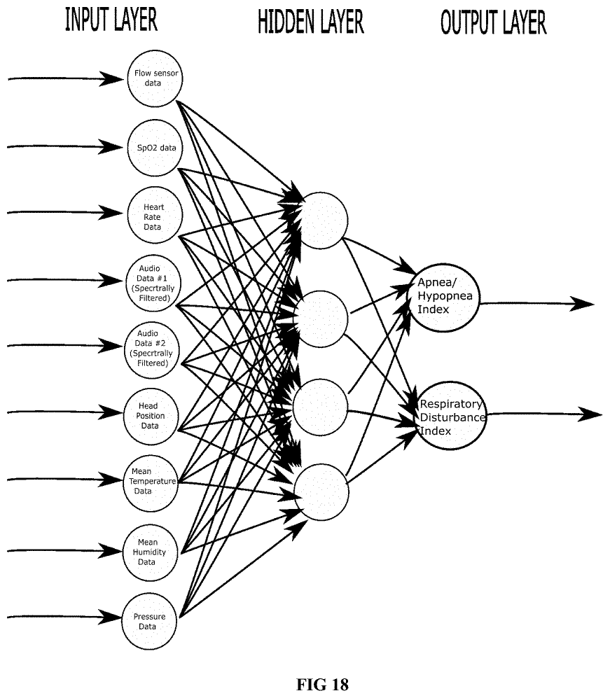

In embodiments, the calculation of these clinically important parameters such as AHI and RDI are obtained using an artificial neural network (ANN) FIG. 18. The ANN is a collection of neurons or nodes which assign weights to the processing of input parameters. These weights are summed to obtain a proportionate representation of output. The weights are obtained by training the ANN using pre-existing data. Such ANNs are well known in the art. The ANN used in one embodiment of this invention FIG. 18 feeds many or all of the input signals obtained from the various sensor technologies onboard the electronics module to the ANN which is subsequently trained to obtain node weightings which relate the input parameters to clinically important output parameters such as AHI and RDI.

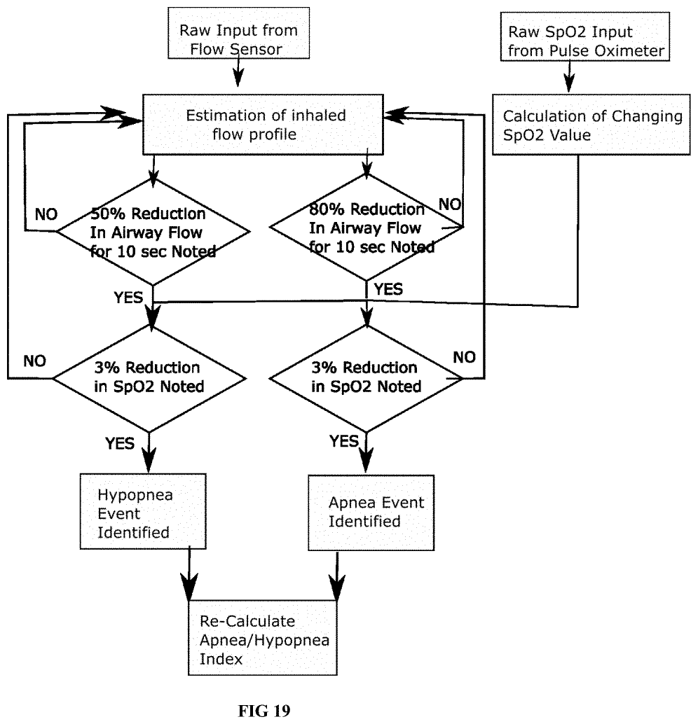

In embodiments of the invention, the calculation of important clinical parameters such as AHI and RDI are obtained using a classical parameter-based or parametric algorithm. In one embodiment of the invention, the parametric algorithm FIG. 19 uses commonly accepted definitions of the clinically important parameters to obtain the parameters. For instance, the calculation of AHI is known in the art to have multiple accepted methods of calculation from those proposed by groups such as the American Academy of Sleep Medicine (AASM) or other methods such as the so-called Chicago Criteria. Typically, an apnea event, for instance, is defined as a drop of 80-100% in inhaled flow accompanied by a transient drop of 3-4% in blood oxygen saturation. Such criteria, varied as they may be, form the basis of a parametric algorithm shown in FIG. 19. It is understood that the algorithm shown in FIG. 19 is exemplary and similar implementations which involve the processing of sensor data into clinically valuable parameters using industry accepted definitions of those parameters should be considered within the scope of this invention. It is further understood that the parametric algorithm as with the ANN, may be performed on either the smart phone APP or on the terminal computer, smart phone or tablet of the health care worker. It is further understood that other common substitutes for the two algorithm types presented here (such as fuzzy logic implementations for instance), should be considered obvious and within the scope of this invention.

In embodiments of the invention, pre-processed and/or processed data such as SpO2, heart rate, mean temperature, mean humidity, head position, breath rate, breath flow, heart rate, biologic indicators and others, received on either the smart phone APP or the external health care workers computer or smart phone or tablet are further processed by way of a predictive analytics algorithm FIG. 22. Such algorithms are known in the art and consist of predicting future outcomes or states based on previously collected data. Such algorithms may be classified as predictive analytics (predicts what is likely to happen) and prescriptive analytics (prescribe what actions to take). The methods for calculation are numerous and may include data mining, Monte-Carlo simulation, pattern identification, and root cause analysis to name a few. In embodiments, predictive and/or prescriptive analytics algorithms may be used to predict patient acceptance of, or reaction to changes in mandibular advancement device titration for instance. Similar uses of predictive and/or prescriptive analytics should be considered obvious and within the scope of this invention.

In embodiments of the invention, a speaker or haptic device is integrated into the electronics module may be used in combination with sensor data collected and or in combination with clock data available on the microprocessor, to alarm the patient of wake-up time, or aberrant measured physiologic or biologic parameters or conditions. For example, an alarm may wake the patient should the patient's blood oxygen saturation drop by 5% or more, ostensibly as a safety precaution.

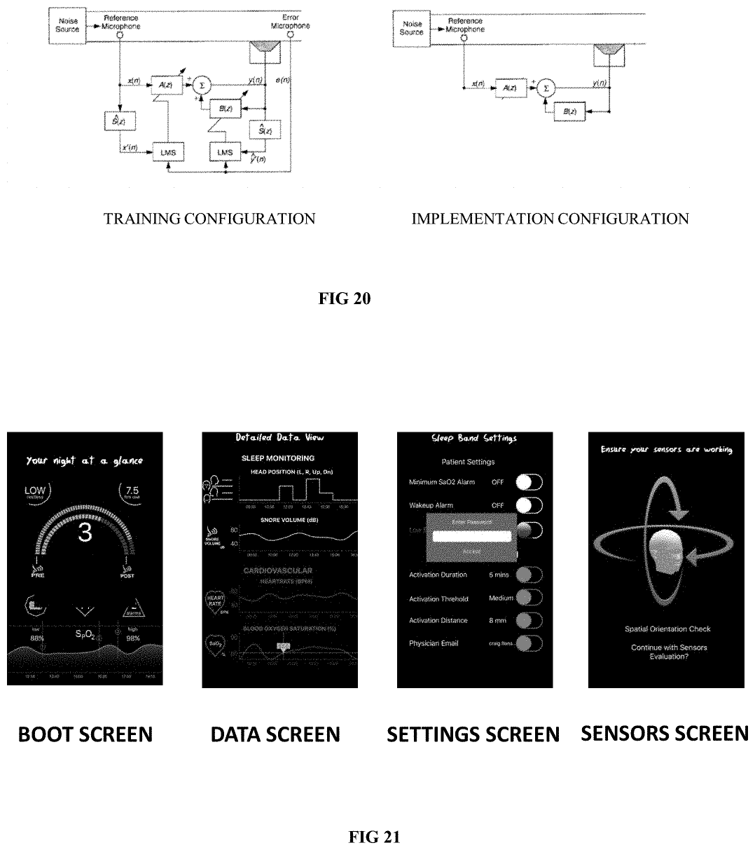

In embodiments, an active noise cancellation (ANC) feature is integrated into the invention, FIG. 20. The ANC feature uses an integral microphone/amplifier and speaker/amplifier in the invention's electronics to pick up snoring sounds and cancel them out using anti-phase sound produced by the speaker. This feature is user settable (with the App) to activate after snoring has been sensed. The ANC feature will remove nighttime noise pollution caused by snoring. The ANC feature requires particular tuning and implementation steps in order to be viable and these steps are described in detail by Flanagan (U.S. Patent Application No. 62/763,086, filed Jun. 4, 2018, entitled COMPILATION OF MANDIBULAR ADVANCEMENT TECHNOLOGIES).

In embodiments, tuning steps for the inventions proposed semi-open loop implementation of an ANC system for intra-oral devices involves four steps: 1. A wide spectrum input signal is output from the microprocessor in the absence of input signal, and the input from the microphone is collected. The secondary path transfer function is then obtained and implemented as a FIR filter placed before the LMS adaptation block of the LMS algorithm.2. Variable amplitude and variable spectral snoring sets are collected and documented. 3. An FxLMS and/or FuLMS configuration is exposed to these signal sets until a set of optimized coefficients are obtained. 4. The coefficients are stored in a table. The implementation steps are now described: 1. The reference microphone is removed from the system. 2. The LMS adaptation block is removed from the algorithm and the B(z) IIR adaptation block (if used) is removed from the algorithm. 3. The algorithm then uses off-line spectral and time domain techniques to determine the amplitude and spectral constituents of the input signal during use to determine which table coefficients to use at any given time. 4. The table looked-up coefficients are used for the S(hat)(z) and/or B(z) transfer functions to determine system output. It should be noted that the semi open loop configuration might be used, or this configuration might just be replaced with an ordinary FuLMS or FxLMS configurations complete with the existence of an error microphone. Either way, the algorithm in combination with the following system elements will constitute a novel implementation of an ANC system.

In embodiments, a magnetic charger which couples to the metal charge stubs of the electronics module will allow for charging of the electronics module between uses.

In embodiments, FIG. 21 shows a representation of user screens on the smart phone dedicated APP which receives data from the intra-oral electronics module and which itself transmits data to the remote computer.

In embodiments, a pressure transducer integral in the electronics module may be used to determine if the hermetic seal of the housing is compromised.

In embodiments, a humidity sensor integral in the electronics module may be used to determine if the hermetic seal of the housing is compromised.

In embodiments, data collected by the invention may be sent to social media sites such as Facebook for display amongst and between fellow patients or patients and health care workers or health care workers only.

We claim:

1. A respiratory monitor system for monitoring sleep disorders such as obstructive sleep apnea, the system comprising: an intra-oral electronics module attached to an oral appliance, a smart phone with dedicated APP, and a remote computer. Said system comprising:
a. a distributed data processing architecture comprised of three processor-based devices in communications with each other, and with devices (1) and (3) communicating through a smart phone (2) intermediary:
1. said intra-oral electronics module,
2. said smart phone with dedicated APP,
3. said remote computer with dedicated program,
b. a scoring algorithm for processing raw intra-oral data into clinical measures of sleep apnea disease state or progression, said scoring algorithm running on either the said smart phone with dedicated APP or the said remote computer.

2. The system of claim 1 wherein the said intra-oral electronics module is removably attached to the oral appliance.

3. The system of claim 1 wherein the said intra-oral electronics module contains a breath flow sensor.

4. The system of claim 1 wherein the said smart phone with dedicated APP processes raw intro-oral data into the apnea/hypopnea index using an apnea disease scoring algorithm.

5. The system of claim 1 wherein the said intra-oral electronics module measures patient device use (compliance) by means of one or a combination of the following techniques:
a. resistivity between exposed terminals on the module housing,
b. measures of head movement,
c. measures of breathing flow,
d. measures of patient sounds,
e. measures of pulse-oximetry data,
f. measures of mean humidity, mean pressure or mean temperature,
g. measures of biologic indicators of disease state,

6. The system of claim 1 wherein the said intra-oral electronics module is divided into two or more devices and wherein the divided devices communicate with each other using mesh networking techniques.

7. The system of claim 1 wherein a predictive analytics algorithm for sleep apnea therapeutic use is integrated into either said smart phone with dedicated APP or said remote computer.

8. A method for monitoring a patient undergoing oral device-based sleep apnea therapy, the method comprising:
a. receiving intra-oral sensor data from one or more sensors configured to track both patient sleep performance, and therapeutic compliance,
b. relaying sensor data, by bi-directional means, to a smart phone with a dedicated APP running an apnea disease scoring algorithm,
c. further relaying the data by bi-directional means from said smart phone with a dedicated APP to a remote computer, smart phone or tablet for display of therapeutic data to health care workers.

9. The method of claim 8 further comprising a means of calculating the apnea/hypopnea index using the said apnea disease scoring algorithm.

10. The method of claim 8 further comprising a means of calculating future disease states or future therapeutic intervention suggestions using a predictive analytics algorithm running on either said smart phone with dedicated APP or said remote computer.

11. The method of claim 8 comprising a means of measuring breath gas flow.

12. The method of claim 8 comprising a means of measuring biologic indicators of sleep apnea disease state.

13. The method of claim 8 comprising a means of measuring patient therapeutic compliance by means of one or a combination of the following techniques:
a. resistivity between exposed terminals on an electronics module housing,
b. measures of head movement,
c. measures of breathing flow,
d. measures of patient sounds,
e. measures of pulse-oximetry data,
f. measures of mean humidity, mean pressure or mean temperature,
g. measures of biologic indicators of disease state,

14. A respiratory monitor system for monitoring sleep disorders such as obstructive sleep apnea, the system comprising:
a. three or more processor-based devices in communications with each other, said devices comprising an intra-oral electronics module, a smart phone with dedicated APP, and remote computer.
i. said intra-oral module itself comprised of two or more intra-oral electronic sub-modules, all sub-modules equipped with sensing and processing means, and all sub-modules in wireless communications with each other.
ii. Said intra-oral sub-modules comprising nodes in a mesh network,

15. The respiratory monitor system of claim 14 wherein the said intra-oral electronics sub-modules are removably attached to the oral appliance.

16. The respiratory system of claim 14 wherein at least one of the said intra-oral electronics sub-modules consists of a breath flow sensor.

17. The respiratory system of claim 14 wherein at least one of the said intra-oral electronics sub-modules senses a biologic indicator of sleep apnea disease state.

18. The respiratory system of claim 14 wherein at least one of the said intra-oral electronics sub-modules measures patient device use (compliance) by means of one or a combination of the following techniques:
a. resistivity between exposed terminals on the module housing,
b. measures of head movement,
c. measures of breathing flow,
d. measures of patient sounds,
e. measures of pulse-oximetry data,
f. measures of mean humidity, mean pressure or mean temperature,
g. measures of biologic indicators of disease state,

19. The respiratory system of claim 14 wherein a sleep apnea scoring algorithm for processing of raw intra-oral data into clinical measures of sleep apnea disease state or progression is integrated into either said smart phone with dedicated APP or said remote computer.

20. The respiratory system of claim 14 wherein a predictive analytics algorithm for sleep apnea therapeutic use is integrated into either said smart phone with dedicated APP or said remote computer.
